(19) United States
(12) Patent Application Publication
(10) Pub. No.: US 2008/0226727 A1
(43) Pub. Date: Sep. 18, 2008

(54) Water-In-Silicone Oil Emulsion for Use as a Sunscreen Product

(75) Inventors: Lorna M Kessell, North Yorkshire (GB); Julian P Hewitt, Durham (GB)

Correspondence Address: MAYER, BROWN, ROWE & MAW LLP, 71 South Wacker, Chicago, IL, 60606, US

(73) Assignee: IMPERIAL CHEMICAL INDUSTRIES PLC, London (GB)

(21) Appl. No.: 10/574,983

(22) PCT Filed: Sep. 24, 2004
(86) PCT No.: PCT/GB2004/004075

§ 371 Date: May 20, 2008

(30) Foreign Application Priority Data
Oct. 9, 2003 (GB) 0323665.0

(51) Int. Cl. A61K 9/14; A61K 33/24

(52) U.S. Cl. 424/489; 424/617

(57) ABSTRACT

A water-in-silicone oil emulsion contains (i) in the range from 0.1 to 25% by weight of particles of metal oxide having a median particle volume diameter in dispersion in the range from 18 to 32 nm, (ii) 5 to 60% by weight of silicone oil, and (iii) greater than 20% by weight of water. The metal oxide particles are preferably incorporated into the emulsion in the form of an aqueous dispersion. The emulsion exhibits good skin feel, effective UV protection, stability and improved transparency.

### FIELD OF INVENTION

The present invention relates to a water-in-silicone oil emulsion comprising particles of metal oxide, a method of making such an emulsion, and in particular to the use of such emulsions as a sunscreen product.

### BACKGROUND

Water-in-oil and oil-in-water emulsions containing particulate metal oxide, such as titanium dioxide, are known and have been used as sunscreen products. Due to the increased awareness of the link between ultraviolet light and skin cancer, there has been an increasing requirement for ultraviolet light protection in everyday skincare and cosmetics products. These products are required to have good emulsion stability, effective UV absorption properties, be transparent in use, and have a pleasant skin feel, and in particular not to feel greasy or oily on the skin. It is an extremely challenging problem to provide a product with all of these properties. There is a constant need in the market place to improve these properties, particularly transparency and skin feel.

### REVIEW OF THE PRIOR ART

EP-0456459 is directed to a water-in-silicone oil emulsion containing a 2-hydroxyalkanoic acid.

### SUMMARY OF THE INVENTION

We have now surprisingly discovered an improved emulsion, which overcomes or significantly reduces at least one of the aforementioned problems.

Accordingly, the present invention provides a water-in-silicone oil emulsion comprising (i) in the range from 0.1 to 25% by weight of particles of metal oxide having a median particle volume diameter in dispersion in the range from 18 to 32 nm, (ii) 5 to 60% by weight of silicone oil, and (iii) greater than 20% by weight of water.

The present invention also provides a process for preparing a water-in-silicone oil emulsion which comprises mixing an aqueous dispersion comprising metal oxide particles having a median particle volume diameter in dispersion in the range from 18 to 32 nm, with a silicone oil under conditions in which a water-in-silicone oil emulsion is formed.

The invention further provides the use of an aqueous dispersion comprising metal oxide particles having a median particle volume diameter in dispersion in the range from 18 to 32 nm, to form a water-in-silicone oil emulsion.

The invention still further provides the use of an aqueous dispersion of metal oxide particles having a median particle volume diameter in dispersion in the range from 18 to 32 nm, in the manufacture of an emulsion having improved skin feel.

Any suitable silicone oil can be used in the water-in-silicone oil emulsion, a principal requirement being cosmetic acceptability. The silicone oil may be at least one volatile or at least one non-volatile silicone oil, or a mixture of volatile and non-volatile silicone oils. The emulsion preferably comprises at least one non-volatile silicone oil, and more preferably the concentration of non-volatile silicone oils is greater than the concentration of volatile silicone oils. In one preferred embodiment, substantially no volatile silicone oils are present in the emulsion.

Suitable silicone oils include those shown in the following table;

| INCI Name | Other Technical Names |
|---|---|
| Cyclomethicone | Methylcyclopolysiloxane |
| Cyclotrisiloxane | Hexamethylcyclotrisiloxane |
| Cyclotetrasiloxane | Octamethylcyclotetrasiloxane |
| Cyclopentasiloxane | Decamethylcyclopentasiloxane |
| Cyclohexasiloxane | Dodecamethylcyclohexasiloxane |
| Cycloheptasiloxane | Tetradecamethylcycloheptasiloxane |
| Dimethicone | Dimethylpolysiloxane, Dimethyl silicone, |
| | Highly polymerized methyl polysiloxane, |
| | Methyl polysiloxane |
| Diphenyl dimethicone | |
| Lauryl dimethicone | |
| Phenyl dimethicone | |
| Trimethylsiloxysilicate | |
| Diisostearyl | |
| trimethylolpropane | |
| siloxy silicate | |
| Dilauroyl trimethylolpropane | |
| siloxy silicate | |
| Phenyl trimethicone | Phenyltris(trimethylsiloxy)silane |
| Triphenyl trimethicone | |
| Disiloxane | Hexamethyldisiloxane, Bis(trimethylsilyl) |
| | ether |
| Dimethiconol | Dihydroxypolydimethylsiloxane |

Preferred silicone oils include dimethylpolysiloxane, dimethyl silicone, highly polymerized methyl polysiloxane, and methyl polysiloxane, known generically as dimethicone. Particularly preferred silicone oils are cyclic oligomeric dialkylsiloxanes, such as the cyclic oligomers of dimethylsiloxane known generically as cyclomethicone.

The silicone oil preferably has a kinematic viscosity of less than 10,000, more preferably less than 5,000, and particularly less than 1,000 cSt. The concentration of silicone oil in an emulsion according to the present invention is suitably in the range from 5 to 60%, preferably 10 to 50%, more preferably 15 to 40%, particularly 20 to 35%, and especially 25 to 30% by weight of the emulsion.

The concentration of water in the emulsion is suitably greater than 20%, preferably in the range from 40 to 90%, more preferably 50 to 80%, particularly 60 to 75%, and especially 65 to 70% by weight of the emulsion.

The metal oxide used in the present invention preferably comprises an oxide of titanium, zinc or iron, and more preferably is titanium dioxide.

The preferred titanium dioxide particles comprise anatase and/or rutile crystal form. The titanium dioxide in the particles preferably comprises a major portion of rutile, more preferably greater than 60% by weight, particularly greater than 70%, and especially greater than 80% by weight of rutile.

The particles of metal oxide used in the present invention are preferably hydrophobic. The hydrophobicity of the metal oxide can be determined by pressing a disc of metal oxide powder, and measuring the contact angle of a drop of water placed thereon, by standard techniques known in the art. The contact angle of a hydrophobic metal oxide is preferably greater than 50°.

The metal oxide particles are preferably coated in order to render them hydrophobic. Suitable coating materials are water-repellent, preferably organic, and include fatty acids, preferably fatty acids containing 10 to 20 carbon atoms, such as lauric acid, stearic acid and isostearic acid, salts of the above fatty acids such as sodium salts and aluminium salts, fatty alcohols, such as stearyl alcohol, and silicones such as polydimethylsiloxane and substituted polydimethylsiloxanes, and reactive silicones such as methylhydrosiloxane and polymers and copolymers thereof. Stearic acid and/or salt thereof is particularly preferred.

The metal oxide particles are preferably treated with up to 25%, more preferably in the range from 3 to 20%, particularly 6 to 17%, and especially 10 to 15% by weight of organic material, preferably fatty acid, calculated with respect to the metal oxide core particles.

The particles of metal oxide may also have an inorganic coating. For example, metal oxide particles, such as titanium dioxide, may be coated with oxides of other elements such as oxides of aluminium, zirconium or silicon, or mixtures thereof such as alumina and silica as disclosed in GB-2205088-A, the teaching of which is incorporated herein by reference. The preferred amount of inorganic coating is in the range from 2 to 25%, more preferably 4 to 20%, particularly 6 to 15%, and especially 8 to 12% by weight, calculated with respect to the weight of metal oxide core particles.

In a preferred embodiment of the invention, the metal oxide particles are coated with both an inorganic and an organic coating, either sequentially or as a mixture. It is preferred that the inorganic coating, preferably alumina, is applied first followed by the organic coating, preferably fatty acid and/or salt thereof. Thus, preferred metal oxide particles for use in the present invention comprise (i) in the range from 60 to 98%, more preferably 65 to 95%, particularly 70 to 80%, and especially 72 to 78% by weight of metal oxide, preferably titanium dioxide, with respect to the total weight of the particles, (II) in the range from 0.5 to 15%, more preferably 2 to 12%, particularly 5 to 10%, and especially 6 to 9% by weight of inorganic coating, preferably alumina, with respect to the total weight of the particles, and (iii) in the range from 1 to 21%, more preferably 4 to 18%, particularly 7 to 15%, and especially 9 to 12% by weight of organic coating, preferably fatty acid and/or salt thereof, with respect to the total weight of the particles.

The individual or primary metal oxide particles are preferably acicular in shape and have a long axis (maximum dimension or length) and short axis (minimum dimension or width). The third axis of the particles (or depth) is preferably approximately the same dimensions as the width. The mean length by number of the metal oxide particles (measured as described herein) is suitably in the range from 50 to 90 nm, preferably 55 to 85 nm, more preferably 60 to 80 nm, particularly 65 to 77 nm, and especially 69 to 73 nm. The mean width by number of the particles (measured as described herein) is suitably in the range from 5 to 20 nm, preferably 8 to 19 nm, more preferably 10 to 18 nm, particularly 12 to 17 nm, and especially 14 to 16 nm. The primary metal oxide particles preferably have a mean aspect ratio d₁:d₂ (where d₁ and d₂, respectively, are the length and width of the particle) in the range from 2 to 8:1, more preferably 3 to 6.5:1, particularly 4 to 6:1, and especially 4.5 to 5.5:1.

The primary metal oxide particles suitably have a median volume particle diameter (equivalent spherical diameter corresponding to 50% of the volume of all the particles, read on the cumulative distribution curve relating volume % to the diameter of the particles—often referred to as the “D(v,0.5)” value), measured as herein described, in the range from 20 to 35 nm, preferably 23 to 33 nm, more preferably 25 to 31 nm, particularly 25 to 28, and especially 25 to 26 nm.

When formed into a dispersion, the particulate metal oxide used in the present invention suitably has a median volume particle diameter (equivalent spherical diameter corresponding to 50% of the volume of all the particles, read on the cumulative distribution curve relating volume % to the diameter of the particles—often referred to as the “D(v,0.5)” value)) (hereinafter referred to as dispersion particle size), measured as herein described, in the range from 18 to 32 nm, preferably 21 to 30 nm, more preferably 23 to 29 nm, particularly 24 to 28 nm, and especially 25 to 27 nm.

The size distribution of the metal oxide particles in dispersion can also be an important parameter in obtaining an emulsion having the required properties. The metal oxide particles in dispersion suitably have less than 16% by volume of particles having a volume diameter of less than 10 nm, preferably less than 6 nm, more preferably less than 4 nm, particularly less than 3 nm, and especially less than 2 nm below the median volume particle diameter. In addition, the metal oxide particles suitably have less than 30% by volume of particles having a volume diameter of less than 6 nm, preferably less than 4 nm, more preferably less than 3 nm, particularly less than 2 nm, and especially less than 1.5 nm below the median volume particle diameter.

Further, the metal oxide particles in dispersion suitably have more than 95% by volume of particles having a volume diameter of less than 55 nm, preferably less than 40 nm, more preferably less than 30 nm, particularly less than 25 nm, and especially less than 20 nm above the median volume particle diameter. In addition, the dispersion metal oxide particles suitably have more than 84% by volume of particles having a volume diameter of less than 13 nm, preferably less than 10 nm, more preferably less than 7 nm, particularly less than 5 nm, and especially less than 3 nm above the median volume particle diameter. Also, the dispersion metal oxide particles suitably have more than 70% by volume of particles having a volume diameter of less than 5 nm, preferably less than 4 nm, more preferably less than 3 nm, particularly less than 2 nm, and especially less than 1.5 nm above the median volume particle diameter.

In a further preferred embodiment of the present invention, the mode volume particle diameter of the metal oxide particles in dispersion suitably account for at least 5.0%, preferably at least 6.0%, more preferably at least 7.0%, particularly in the range from 8.0 to 10.0%, and especially 8.5 to 9.5% by volume of the total metal oxide particles.

It is preferred that none of the metal oxide particles in dispersion should have an actual particle size exceeding 200 nm. Particles exceeding such a size may be removed by milling processes which are known in the art. However, milling operations are not always totally successful in eliminating all particles greater than a chosen size. In practice, therefore, the size of 95%, preferably 99% by volume of the particles should not exceed 200 nm, preferably 150 nm.

The dispersion particle size of the metal oxide particles described herein may be measured by electron microscope, coulter counter, sedimentation analysis and static or dynamic light scattering. Techniques based on sedimentation analysis are preferred. The median particle size may be determined by plotting a cumulative distribution curve representing the percentage of particle volume below chosen particle sizes and measuring the 50th percentile. The median particle volume diameter of the dispersion metal oxide particles is suitably measured using a Brookhaven particle sizer, as described herein. The particle size distributions, including the mode, can also be obtained from the same cumulative distribution curve.

In a particularly preferred embodiment of the invention, the metal oxide particles have a BET specific surface area, measured as described herein, of greater than 40, more preferably in the range from 50 to 100, particularly 60 to 90, and especially 65 to 75 m²/g.

The metal oxide particles used in the present invention exhibit improved transparency suitably having an extinction coefficient at 524 nm (E₅₂₄), measured as herein described, of less than 2.0, more suitably less than 1.5, preferably less than 1.2, more preferably in the range from 0.1 to 1.0, particularly 0.2 to 0.9, and especially 0.3 to 0.8 l/g/cm. In addition, the metal oxide particles suitably have an extinction coefficient at 450 nm (E₄₅₀), measured as herein described, of less than 4.0, preferably in the range from 0.2 to 3.0, more preferably 0.4 to 2.5, particularly 0.6 to 2.0, and especially 0.8 to 1.5 l/g/cm.

The metal oxide particles exhibit effective UV absorption, suitably having an extinction coefficient at 360 nm (E₃₆₀), measured as herein described, of greater than 3.0, preferably in the range from 4.0 to 12.0, more preferably 5.0 to 10.0, particularly 6.0 to 9.0, and especially 7.0 to 8.0 l/g/cm. The metal oxide particles also suitably have an extinction coefficient at 308 nm (E₃₀₈), measured as herein described, of greater than 30, preferably in the range from 35 to 65, more preferably 40 to 60, particularly 45 to 55, and especially 46 to 50 l/g/cm.

The metal oxide particles suitably have a maximum extinction coefficient E(max), measured as herein described, of greater than 45, preferably in the range from 50 to 80, more preferably 55 to 75, particularly 60 to 70, and especially 65 to 70 l/g/cm. The metal oxide particles suitably have a % (max), measured as herein described, in the range from 260 to 290, preferably 265 to 287, more preferably 270 to 285, particularly 275 to 283, and especially 279 to 281 nm.

In a particularly preferred embodiment, the metal oxide is incorporated into the emulsion of the present invention in the form of an aqueous dispersion. By dispersion is meant a true dispersion, i.e. where the solid particles are stable to aggregation. The particles in the dispersion are relatively uniformly dispersed and resistant to settling out on standing, but if some settling out does occur, the particles can be easily redispersed by simple agitation. The aqueous dispersion can be prepared by milling the metal oxide with a particulate grinding medium in water, preferably in the presence of a suitable dispersing agent.

The concentration of metal oxide particles in the aqueous dispersion is suitably at least 25%, preferably at least 35%, more preferably at least 40%, particularly at least 45%, especially at least 50% by weight of the dispersion.

The aqueous dispersion preferably comprises in the range from 2 to 15%, more preferably 4 to 12%, particularly 5 to 10%, and especially 6 to 8% by weight of the dispersion, of a dispersing agent, preferably a non-ionic surfactant.

In a preferred embodiment, the non-ionic surfactant has a HLB (Hydrophile Lipophile Balance) value of greater than 3, suitably in the range from 5 to 20, preferably 7 to 18, more preferably 9 to 16, particularly 11 to 14, and especially 12 to 13. The molecular weight (number average) of the non-ionic surfactant is suitably in the range from 150 to 2000, preferably 200 to 1500, more preferably 250 to 800, particularly 300 to 600, and especially 350 to 450.

Suitable materials include alkoxylate surfactants, and surfactants that can be derived from natural materials such as fatty acid esters, ethers, hemi-acetals or acetals of polyhydroxylic compounds or a fatty acid amide which is N-substituted with the residue of a polyhydroxylic compound.

The term alkoxylate surfactant is used to refer to compounds in which a hydrophobe, usually a hydrocarbyl group, is connected through the residue of a linking group normally having a reactive hydrogen atom to an oligomeric or polymeric chain of alkylene oxide residues. The hydrocarbyl group is typically a chain, optionally branched, preferably an alkyl chain, suitably comprising in the range from 5 to 54, preferably 6 to 36, more preferably 7 to 20, particularly 8 to 15, and especially 9 to 11 carbon atoms. The linking group can be an oxygen atom (hydroxyl group residue); a carboxyl group (fatty acid or ester residue); an amino group (amine group residue); or a carboxyamido (carboxylic amide residue). The alkylene oxide residues are typically residues of ethylene oxide (C₂H₄O) or propylene oxide (C₃H₆O) or combinations of ethylene and propylene oxide residues. When combinations are used the proportion of ethylene oxide residues are preferably at least about 50 mole %, and more preferably at least 75 mole %, the remainder being propylene oxide residues. In a particularly preferred embodiment of the invention, substantially all the residues are ethylene oxide residues. The number of alkylene oxide, preferably ethylene oxide, residues in the surfactant molecule is suitably less than 100, preferably in the range from 2 to 50, more preferably 3 to 25, particularly 4 to 15, and especially 5 to 8.

Examples of suitable alkoxylate surfactants include alcohol alkoxylates, of the formula (Ia): R¹—O-(AO)ₙ-H; a fatty acid alkoxylate of the formula (Ib): R¹—COO-(AO)ₙ-R² (plus co-products); a fatty amine alkoxylate of the formula (Ic): R¹—NR³-(AO)ₙ-H; or a fatty amide alkoxylate of the formula (Id); R¹—NR³-(AO)ₙ-H, where each R¹ is independently preferably a C₆ to C₂₀, more preferably C₇ to C₁₅, particularly C₈ to C₁₂, and especially C₉ to C₁₁, hydrocarbyl, optionally branched, preferably alkyl group; R² is a hydrogen atom or a C₁ to C₆ alkyl group; and each R³ is independently a C₁ to C₆ alkyl group or a group (AO)ₙ-H; each AO is independently an ethylene oxide or propylene oxide, preferably ethylene oxide group; and the total of the indices n in the molecule is preferably in the range from 2 to 25, more preferably 3 to 15, particularly 4 to 10, and especially 5 to 7. Alkyl phenyl ethoxylates could also be used, but these are generally not now desired in personal care and cosmetic products for other reasons and are thus not usually used in the present invention. In a particularly preferred embodiment of the invention, the non-ionic surfactant comprises at least one alcohol alkoxylate as described above.

The concentration of non-ionic surfactant in the aqueous dispersion of metal oxide particles used in the present invention, or in the emulsion according to the present invention, is suitably in the range from 1 to 100%, preferably 5 to 50%, more preferably 10 to 30%, particularly 13 to 25%, and especially 15 to 20% by weight, calculated with respect to the weight of metal oxide particles.

In a preferred embodiment of the invention, a combination of two or more non-ionic surfactants described herein having HLB values within the above preferred ranges is employed. Suitably a mixture of two such surfactants is used which differ in molecular weights (number average), preferably by an amount in the range from 50 to 1500, more preferably 100 to 1000, particularly 250 to 700, and especially 350 to 450. The ratio by weight of the two surfactants in the composition is preferably in the range from 0.2 to 5:1, more preferably 0.5 to 2:1, particularly 0.75 to 1.3:1, and especially 0.9 to 1.1:1. Both of the surfactants are preferably alcohol alkoxylates as described above, with the total amount of surfactants present in the composition being preferably in the ranges given above for the non-ionic surfactant.

In an alternative preferred embodiment of the invention, a combination of two or more non-ionic surfactants is employed, at least one relatively hydrophilic surfactant i.e. preferably having a HLB value of greater than or equal to 9, and at least one relatively hydrophobic surfactant i.e. preferably having a HLB value of less than 9. The hydrophilic surfactant is suitably at least one of the non-ionic surfactants described herein, preferably an alcohol alkoxylate.

The hydrophilic surfactant preferably has a HLB value in the range from 9.5 to 15, more preferably 11 to 14, particularly 11.5 to 13.5, and especially 12 to 13.

The hydrophobic surfactant may also be at least one of the non-ionic surfactants described herein, also suitably an alcohol alkoxylate, and preferably has a HLB value in the range from 2 to 8.5, more preferably 3 to 7.5, particularly 4 to 7, and especially 5 to 6.

When a combination of hydrophilic and hydrophobic surfactants is used, the total amount of hydrophilic and hydrophobic surfactants present in the aqueous dispersion is preferably in the ranges given above for the non-ionic surfactant. The ratio by weight of the at least one hydrophilic surfactant to the at least one hydrophobic surfactant is preferably in the range from 0.1 to 10:1, more preferably 0.3 to 3.3:1, particularly 0.6 to 1.7:1, and especially 0.8 to 1.2:1.

The aqueous dispersion preferably comprises at least one defoaming agent. Standard defoaming agents known in the art may be employed, preferably silicone defoaming agents. The concentration of defoaming agent present in the aqueous dispersion is preferably in the range from 0.1 to 5%, more preferably 0.5 to 2%, and particularly 0.8 to 1.2% by weight of the dispersion. The aqueous dispersion may also comprise other standard ingredients used in the art, for example preservatives.

The water-in-silicone oil emulsion preferably comprises at least one emulsifier. The concentration of emulsifiers is preferably in the range from 0.1 to 10%, more preferably 0.25 to 5%, particularly 0.5 to 3%, and especially 0.8 to 2% by weight of the emulsion.

In a preferred embodiment, the HLB value of the emulsifier system employed is preferably less than 10, more preferably less than 8, particularly less than 6, and especially less than 5.

Silicone-based emulsifiers are preferred, such as alkoxylated derivatives of siloxane polymers. Particularly preferred emulsifiers comprise both ethylene oxide and propylene oxide groups, for example suitable commercially available materials include PEG/PPG-17/18 dimethicone (Dow Corning 2-5220 Resin Modifier), lauryl PEG/PPG-18/18 methicone (Dow Corning 5200 Formulation Aid), PEG/PPG-18/18 dimethicone (Dow Corning 3225c Formulation Aid, Dow Corning 5225c Formulation Aid, Dow Corning 190 Surfactant), bis-PEG/PPG-20/20 dimethicone (Abil B8832, ex Degussa), cetyl PEG/PPG-10/1 dimethicone (Abil EM90, ex Degussa), and bis-PEG/PPG-14/14 dimethicone (Abil EM97, ex Degussa).

In a particularly preferred embodiment of the invention, the emulsion comprises less than 10%, more preferably less than 5%, particularly less than 2%, and especially less than 1% by weight of the emulsion of any oil or emollient other than silicone oil. These other oils or emollients include paraffin oils, branched hydrocarbons, triglyceride esters, esters of fatty acids and fatty alcohols, vegetable oils, or alkoxylated fatty alcohol ethers.

The emulsion is preferably substantially free of any oil or emollient other than silicone oil, i.e. silicone oil is preferably the sole oil or emollient present in the emulsion. A surprising feature of the present invention is that properties such as good emulsion stability, effective UV absorption, transparency in use, and/or pleasant skin feel can be achieved with an emulsion containing silicone oil as the sole oil or emollient.

The water-in-silicone oil emulsions are prepared by mixing an aqueous phase with a silicone oil phase under such conditions that a water-in-silicone oil emulsion is formed. The emulsion is preferably made by initially mixing the silicone oil with at least one emulsifier and, when desired, any other oleophilic components to form a silicone oil phase. This oil phase is subsequently mixed with an aqueous dispersion of metal oxide to form a water-in-silicone oil emulsion. Alternatively, the metallic oxide, preferably in the form of an aqueous dispersion, may be mixed with an emulsion which has previously been prepared by mixing a silicone oil phase containing emulsifier with an aqueous phase.

Such water-in-silicone oil emulsions may be prepared at room temperature, or alliteratively elevated temperatures, for example above 40° C., may be employed. When components which are solid at room temperature are present, it is usually necessary to heat one or both phases before mixing.

The water-in-silicone oil emulsion according to the present invention suitably exhibits reduced whiteness, preferably having a change in whiteness ΔL, measured as herein described, of less than 3, more preferably less than 2.5, particularly less than 2.0, and especially less than 1.5. In addition, the emulsion suitably has a whiteness index, measured as herein described, of less than 100%, preferably in the range from 10 to 90%, more preferably 20 to 80%, particularly 30 to 70%, and especially 40 to 60%.

The water-in-silicone oil emulsion suitably has a Sun Protection Factor (SPF), measured as herein described, of greater than 5, preferably greater than 10, more preferably greater than 15, particularly greater than 20, and especially greater than 25.

The water-in-silicone oil emulsion preferably exhibits improved skin feel compared to conventional emulsions containing metal oxide in the aqueous phase. Such conventional emulsions exhibit a predominantly “waxy” skin feel, with a low degree of lubricity. Emulsions according to the present invention surprisingly have improved skin feel, measured as herein described, for example conferring a significantly higher degree of lubricity to the skin.

The water-in-silicone oil emulsion preferably exhibits stability against emulsion separation, i.e. the emulsion remains homogeneous when stored for extended periods at various temperatures. The emulsion is preferably stable, measured as herein described, at 5° C., more preferably at 25° C., particularly at 40° C., and especially at 50° C., preferably for at least one month, more preferably at least 2 months, and particularly for at least 3 months.

The particulate metal oxide as defined herein, may provide the only ultraviolet light attenuators in an emulsion according to the invention, but other sunscreening agents, such as other metal oxides and/or other organic materials may also be added. For example, the preferred titanium dioxide particles defined herein may be used in combination with other existing commercially available titanium dioxide and/or zinc oxide sunscreens. Suitable organic sunscreens for use with a composition according to the invention include p-methoxy cinnamic acid esters, salicylic acid esters, p-amino benzoic acid esters, non-sulphonated benzophenone derivatives, derivatives of dibenzoyl methane and esters of 2-cyanoacrylic acid. Specific examples of useful organic sunscreens include benzophenone-1, benzophenone-2, benzophenone-3, benzophenone-6, benzophenone-8, benzophenone-12, isopropyl dibenzoyl methane, butyl methoxy dibenzoyl methane, ethyl dihydroxypropyl PABA, glyceryl PABA, octyl dimethyl PABA, octyl methoxycinnamate, homosalate, octyl salicylate, octyl triazone, octocrylene, etocrylene, menthyl anthranilate, and 4-methylbenzylidene camphor.

Other ingredients can be added to the emulsion and these ingredients may be introduced in any convenient manner. For example they can be mixed with the emulsion, or added to the aqueous phase, or the silicone oil phase before these components are mixed together. For example, perfumes, antioxidants, moisturizers, thickeners and preservatives are normally added to emulsions to produce a commercially acceptable cosmetic product.

The water-in-silicone oil emulsions according to the present invention find use as sunscreen compositions, as skin protectants, as moisturizers and as decorative cosmetics such as liquid foundations.

In this specification the following test methods have been used:

1) Particle Size Measurement of Primary Metal Oxide Particles

A small amount of metal oxide, typically 2 mg, was pressed into approximately 2 drops of an oil, for one or two minutes using the tip of a steel spatula. The resultant suspension was diluted with solvent and a carbon-coated grid suitable for transmission electron microscopy was wetted with the suspension and dried on a hot-plate. Approximately 18 cm×21 cm photographs were produced at an appropriate, accurate magnification. Generally about 300-500 crystals were displayed at about 2 diameters spacing. A minimum number of 300 primary particles were sized using a transparent size grid consisting of a row of circles of gradually increasing diameter, representing spherical crystals. Under each circle a series of ellipsoid outlines were drawn representing spheroids of equal volume and gradually increasing eccentricity. The basic method assumes log normal distribution standard deviations in the 1.2-1.6 range (wider crystal size distributions would require many more crystals to be counted, for example of the order of 1000). The dispersion method described above has been found to be suitable for producing almost totally dispersed distributions of primary metal oxide particles whilst introducing minimal crystal fracture. Any residual aggregates (or secondary particles) are sufficiently well defined that they, and any small debris, can be ignored, and effectively only primary particles included in the count.

Mean length, mean width and length/width size distributions of the primary metal oxide particles can be calculated from the above measurements. Similarly, the median particle volume diameter of the primary particles can also be calculated.

2) Particle Size Measurement of Metal Oxide Particles in Dispersion

A dispersion of metal oxide particles was produced by mixing 104 g of deionised water, 16 g of isodecyl alcohol 6-ethoxylate, and 80 g of metal oxide. The mixture was passed through a horizontal bead mill, containing zirconia beads as grinding media, operating at approximately 1500 r.p.m. for 15 minutes. The dispersion of metal oxide particles was diluted to between 30 and 40 g/l by mixing with a 0.1% by weight aqueous solution of isodecyl alcohol 6-ethoxylate. The diluted sample was analysed on the Brookhaven BI-XDC particle sizer in centrifugation mode, and the median particle volume diameter, particle size distribution and mode (in a 1 nm band width) particle diameter % determined.

3) BET Specific Surface Area of Metal Oxide Particles

The single point BET specific surface area was measured using a Micromeritics Flowsorb II 2300.

4) Extinction Coefficients

An aqueous dispersion of metal oxide particles (for example as described in 2) above) was diluted with 100 ml of water (or 0.1% by weight aqueous solution of isodecyl alcohol 6-ethoxylate). This diluted sample was then further diluted with water (or the above surfactant solution) in the ratio sample:water (or surfactant solution) of 1:19. The total dilution was 1:20,000. The diluted sample was then placed in a spectrophotometer (Perkin-Elmer Lambda 2 UV/VIS Spectrophotometer) with a 1 cm path length, and the absorbance of UV and visible light measured. Extinction coefficients were calculated from the equation A=E.c.l, where A=absorbance, E=extinction coefficient in litres per gram per cm, c=concentration in grams per litre, and l=path length in cm.

5) Change in Whiteness and Whiteness Index

A water-in-silicone oil emulsion (for example a basic recipe comprising 7.5% by weight of DC 749 Fluid (ex Dow Corning), 7.5% by weight of DC 200 Fluid, 5cs (ex Dow Corning), 10.0% by weight of DC 5225c Formulation Aid (ex Dow Corning), 63.5% by weight of water, 4.0% by weight of glycerine BP, 1.0% by weight of sodium chloride, 0.5% by weight of Germaben II (ex ISP), and 6.0% by weight of particulate metal oxide) was coated on to the surface of a glossy black card and drawn down using a No 2 K bar to form a film of 12 μm wet thickness. The film was allowed to dry at room temperature for 10 minutes and the whiteness of the coating on the black surface (L_{F}) measured using a Minolta CR300 colorimeter. The change in whiteness ΔL was calculated by subtracting the whiteness of the substrate (L_{S}) from the whiteness of the coating (L_{F}). The whiteness index is the percentage change in whiteness ΔL compared to a standard titanium dioxide (=100% value) (Tayca MT100T (ex Tayca Corporation)).

6) Sun Protection Factor

The Sun Protection Factor (SPF) of a sunscreen formulation was determined using the in vitro method of Diffey and Robson, J. Soc. Cosmet. Chem. Vol. 40, pp 127-133, 1989.

7) HLB Value

The HLB value of the surfactants was calculated according to the method of Schick, “Non-Ionic Surfactants”, Surf. Sci. Series Vol. 1, Chapter 18.

8) Skin Feel

Skin feel was measured by trained assessors, using a protocol in which test samples were assigned % numerical scores for a series of sensory attributes. 2 ml of the emulsion was spread over a circular area, approximately 5 cm in diameter, on the inner forearm. After spreading, the afterfeel of the test sample on the skin was characterized in the following terms:—% “oily” skin feel; % “waxy” skin feel; % “greasy” skin feel; and % “silicone” skin feel.

9) Stability

Stability was measured by storing samples of the emulsion in screw-top glass jars at temperatures of 5° C., 25° C., 40° C., and 50° C. Samples were assessed visually after 1, 2 and 3 months for any signs of inhomogeneity or emulsion separation.

The invention is illustrated by the following non-limiting examples.

### EXAMPLES

### Example 1

A water-in-silicone oil emulsion was prepared from the following ingredients;

| | % by weight |
|---|---|
| Phase A | |
| DC 749 Fluid (ex Dow Corning) | 7.5 |
| DC 200 Fluid, 5cs (ex Dow Corning) | 7.5 |
| DC 5225c Formulation Aid (ex Dow Corning) | 10 |
| Phase B | |
| Water; pure | 54.5 |
| Glycerine BP | 4 |
| Sodium Chloride | 1 |
| Solaveil CT 10W (trade mark, ex Uniqema) | 15 |
| (40% by weight aqueous dispersion of titanium dioxide) | |
| Germaben II (ex ISP) | 0.5 |

The ingredients of aqueous phase B were combined with high-shear mixing. The ingredients of phase A were mixed with stirring. Phase B was slowly added to phase A with intensive mixing, followed by mixing with an Ultra-Turrax high-shear mixer for 1 minute. The mixture was stirred intensively for a further 30 minutes.

The water-in-silicone oil emulsion exhibited good skin feel (after feel was assessed as 10% “oily”, 20% “waxy”, 5% “greasy”, and 65% “silicone-like”), a change in whiteness ΔL of −0.61, and a Sun Protection Factor of 11.6. The emulsion was stable at all temperatures for at least one month, with no visible signs of separation.

### Example 2

A water-in-silicone oil emulsion was prepared, using the procedure as described in Example 1, from the following ingredients;

| | % by weight |
|---|---|
| Phase A | |
| DC 9040 Silicone Elastomer Blend (ex Dow Corning) | 10 |
| DC 345 Fluid (ex Dow Corning) | 10 |
| DC 5225c Formulation Aid (ex Dow Corning) | 10 |
| Phase B | |
| Water; pure | 52.5 |
| Glycerine BP | 5 |
| Sodium Chloride | 2 |
| Solaveil CT 10W (trade mark, ex Uniqema) | 10 |
| Germaben II (ex ISP) | 0.5 |

The water-in-silicone oil emulsion exhibited good skin feel (after feel was assessed as 0% “oily”, 30% “waxy”, 5% “greasy”, and 65% “silicone-like”), a change in whiteness ΔL of 2.35, and a Sun Protection Factor of 9.2. The emulsion was stable at all temperatures for at least one month, with no visible signs of separation.

The above examples illustrate the improved properties of a water-in-silicone oil emulsion according to the present invention.

1. A water-in-silicone oil emulsion comprising (i) in the range from 0.1 to 25% by weight of particles of metal oxide having a median particle volume diameter in dispersion in the range from 18 to 32 nm, (ii) 5 to 60% by weight of silicone oil, and (iii) greater than 20% by weight of water.

2. An emulsion according to claim 1 wherein the metal oxide is incorporated into the emulsion in the form of an aqueous dispersion.

3. An emulsion according to claim 1, wherein the metal oxide particles are hydrophobic.

4. An emulsion according to claim 1, wherein the metal oxide particles comprise titanium dioxide.

5. An emulsion according to claim 1 wherein the mean length of the metal oxide particles is in the range from 50 to 90 nm, and the mean width is in the range from 5 to 20 nm.

6. An emulsion according to any claim 1 wherein the metal oxide particles have a median particle volume diameter in dispersion of 23 to 29 nm, preferably 24 to 28 nm.

7. An emulsion according to claim 1 wherein the metal oxide particles in dispersion have (i) less than 16% by volume of particles having a volume diameter of less than 10 nm below the median volume particle diameter, (ii) less than 30% by volume of particles having a volume diameter of less than 6 nm below the median volume particle diameter, (iii) more than 95% by volume of particles having a volume diameter of less than 55 nm above the median volume particle diameter, (iv) more than 84% by volume of particles having a volume diameter of less than 13 nm above the median volume particle diameter, and (v) more than 70% by volume of particles having a volume diameter of less than 5 nm above the median volume particle diameter.

8. An emulsion according to claim 7 wherein the metal oxide particles in dispersion have (i) less than 16% by volume of particles having a volume diameter of less than 4 nm below the median volume particle diameter, (ii) more than 95% by volume of particles having a volume diameter of less than 30 nm above the median volume particle diameter, and (iii) more than 84% by volume of particles having a volume diameter of less than 7 nm above the median volume particle diameter.

9. An emulsion according to claim 1 wherein the metal oxide particles have at least one, and preferably all, of (i) an extinction coefficient at 524 nm of less than 1.5 l/g/cm, (ii) an extinction coefficient at 450 nm in the range from 0.2 to 3.0 l/g/cm, (iii) an extinction coefficient at 360 nm in the range from 4.0 to 12.0 l/g/cm, (iv) an extinction coefficient at 308 nm in the range from 35 to 65 l/g/cm, (v) a maximum extinction coefficient in the range from 50 to 80 l/g/cm, and (vi) a A (max) in the range from 265 to 287 nm.

10. An emulsion according to claim 9 wherein the metal oxide particles have an extinction coefficient at 524 nm in the range from 0.1 to 1.0 l/g/cm.

11. An emulsion according to claim 2 wherein the aqueous dispersion comprises at least 25% by weight of metal oxide particles.

12. An emulsion according to claim 2 wherein the aqueous dispersion comprises in the range from 2 to 15% by weight of at least one dispersing agent.

13. An emulsion according to claim 12 wherein the dispersing agent comprises at least one non-ionic surfactant.

14. An emulsion according to claim 1 comprising in the range from 5 to 50% by weight of at least one non-ionic dispersing agent, calculated with respect to the metal oxide particles.

15. An emulsion according to claim 1 comprising in the range from 0.1 to 10% by weight of at least one emulsifier.

16. An emulsion according to claim 15 wherein the emulsifier comprises a silicone emulsifier.

17. An emulsion according to claim 1 comprising less than 10% by weight of any oil other than silicone oil.

18. An emulsion according to claim 1 wherein silicone oil is the sole oil present.

19. An emulsion according to claim 1 having a change in whiteness ΔL of less than 3, preferably less than 2.5.

20. An emulsion according to claim 1 having a whiteness index in the range from 10 to 90%.

21. A process for preparing a water-in-silicone oil emulsion which comprises mixing an aqueous dispersion comprising metal oxide particles having a median particle volume diameter in dispersion in the range from 18 to 32 nm, with a silicone oil under conditions in which a water-in-silicone oil emulsion is formed.

22. A process according to claim 21 wherein the aqueous dispersion comprises at least 25% by weight of metal oxide particles.

23. The use of an aqueous dispersion comprising metal oxide particles having a median particle volume diameter in dispersion in the range from 18 to 32 nm, to form a water-in-silicone oil emulsion.

24. The use of an aqueous dispersion of metal oxide particles having a median particle volume diameter in dispersion in the range from 18 to 32 nm, in the manufacture of an emulsion having improved skin feel.
